(19) United States
(12) Patent Application Publication
(10) Pub. No.: US 2005/0049640 A1
(43) Pub. Date: Mar. 3, 2005

(54) Electromagnetic fields increase in vitro and in vivo angiogenesis through endothelial release of FGF-2

(76) Inventors: Geoffrey C. Gurtner, New York, NY (US); Oren Tepper, New York, NY (US); Jamie Levine, New York, NY (US)

Correspondence Address: Michael L. Goldman;Nixon Peabody LLP, Clinton Square, P.O. Box 31051, Rochester, NY, 14603-1051, US

(21) Appl. No.: 10/843,141

(22) Filed: May 11, 2004

Related U.S. Application Data
(60) Provisional application No. 60/469,711, filed on May 12, 2003.

(51) Int. Cl. A61N 1/00

(52) U.S. Cl. 607/2

(57) ABSTRACT

The present invention relates to a method of inducing angiogenesis in a cell or tissue by applying an electromagnetic field to the cell or tissue under conditions effective to induce angiogenesis. Also disclosed is a method of treating an ischemic condition in a patient by applying an electromagnetic field to ischemic tissue in a patient under conditions effective to treat the ischemic condition by inducing angiogenesis. A method of tissue engineering is also disclosed. This method involves providing a tissue scaffold and subjecting the tissue scaffold to an electromagnetic field under conditions effective to form a vascularized tissue scaffold. Further disclosed is a method of inducing activity of angiogenic growth factors by applying an electromagnetic field to a cell or tissue under conditions effective to induce activity of an angiogenic growth factor.

This application claims the benefit of U.S. Provisional Patent Application Ser. No. 60/469,711, filed May 12, 2003, which is hereby incorporated by reference in its entirety.

### FIELD OF THE INVENTION

This invention relates to methods of inducing angiogenesis, treating an ischemic condition, tissue engineering, and inducing activity of angiogenic factors by application of electromagnetic fields.

### BACKGROUND OF THE INVENTION

Electromagnetic forces are believed to play a role in the normal repair of human tissues. The therapeutic efficacy of various forms of electrical stimulation, including capacitative coupling, direct current, combined magnetic fields, and pulsed electromagnetic fields (“PEMF”) have been intensely investigated over the past 30 years (Bassett et al., “Augmentation of Bone Repair by Inductively Coupled Electromagnetic Fields,” Science 184:575-577 (1974); Ryaby, “Clinical Effects of Electromagnetic and Electric Fields on Fracture Healing,” Clin. Orthop. S205-15 (1998)). The therapeutic effects of PEMF were first demonstrated in bone by Basset and colleagues, whose reports led to clinical trials and widespread commercial availability. Subsequently, PEMF has been demonstrated in blinded trials to be a safe and effective means of treating non-healing bone fractures (Scott et al., “A Prospective, Double-Blind Trial of Electrical Capacitive Coupling in the Treatment of Non-Union of Long Bones,” J. Bone Joint Surg. Am. 76:820-826 (1994); Sharrard, “A Double-Blind Trial of Pulsed Electromagnetic Fields for Delayed Union of Tibial Fractures,” J. Bone Joint Surg. Br 72:347-355 (1990)).

Despite the clinical evidence demonstrating PEMF to be an effective treatment modality in bone healing, its mechanism of action is unknown. PEMF is able to upregulate several cytokines which are important in promoting osteoblast differentiation during fracture repair, including bone morphogenetic proteins 2 and 4, and transforming growth factor-β (Bostrom et al., “Immunolocalization and Expression of Bond Morphogenetic Proteins 2 and 4 in Fracture Healing,” J. Orthop. Res. 13:357-367 (1995); Bodamyali et al., “Pulsed Electromagnetic Fields Simultaneously Induce Osteogenesis and Upregulate Transcription of Bone Morphogenetic Proteins 2 and 4 in Rat Osteoblasts In Vitro,” Biochem. Biophys. Res. Commun. 250:458-461 (1998)). However, more direct barometers of osteoblast ftunction, such as collagen synthesis, proliferation, alkaline phosphatase activity, and prostaglandin E2 production are not significantly altered in the presence of PEMF (Guerkov et al., “Pulsed Electromagnetic Fields Increase Growth Factor Release by Nonunion Cells,” Clin. Orthop. 265:279 (2001); Lohmann et al., “Pulsed Electromagnetic Field Stimulation of MG63 Osteoblast-Like Cells Affects Differentiation and Local Factor Production,” J. Orthop. Res. 18:637-46 (2000); Aaron et al., “Upregulation of Basal TGFbetal Levels by EMF Coincident with Chondrogenesis—Implications for Skeletal Repair and Tissue Engineering,” J. Orthop. Res. 20:223-240 (2002)). Thus, it seems unlikely that the clinical success of PEMF is entirely attributable to an effect on osteoblasts alone.

This has led investigators to study the effect of PEMF on other processes important during tissue repair (Jasti et al., “Effect of a Wound Healing Electromagnetic Field on Inflammatory Cytokine Gene Expression in Rats,” Biomed. Sci. Instrum. 37:209-214 (2001)). Angiogenesis, the sprouting of new blood vessels, is critical for successful fracture healing (Steinbrech et al., “VEGF Expression in an Osteoblast-Like Cell Line is Regulated by a Hypoxia Response Mechanism,” Am. J. Physiol. Cell. Physiol. 278:C853-860 (2000); Donski et al., “Growth in Revascularized Bone Grafts in Young Puppies,” Plast. Reconstr. Surg. 65:239-243 (1979)), but the effects of PEMF on angiogenesis are not well understood (Yen-Patton et al., “Endothelial Cell Response to Pulsed Electromagnetic Fields: Stimulation of Growth Rate and Angiogenesis In Vitro,” J. Cell Physiol. 134:37-46 (1988)).

The present invention is directed to overcoming these deficiencies in the art.

### SUMMARY OF THE INVENTION

One aspect of the present invention relates to a method of inducing angiogenesis in a cell or tissue. This method involves applying an electromagnetic field to the cell or tissue under conditions effective to induce angiogenesis.

Another aspect of the present invention relates to a method of treating an ischemic condition in a patient. This method involves applying an electromagnetic field to ischemic tissue in a patient under conditions effective to treat the ischemic condition by inducing angiogenesis.

A further aspect of the present invention relates to a method of tissue engineering. This method involves providing a tissue scaffold and subjecting the tissue scaffold to an electromagnetic field under conditions effective to form a vascularized tissue scaffold.

Yet another aspect of the present invention relates to a method of inducing activity of angiogenic growth factors. This method involves applying an electromagnetic field to a cell or tissue under conditions effective to induce activity of an angiogenic growth factor.

### BRIEF DESCRIPTION OF THE DRAWINGS

FIGS. 1A-H show PEMF stimulation of 3-dimensional angiogenesis in vitro. FIG. 1A shows the results of a 3-dimensional angiogenesis assay performed on Human Umbilical Vein Endothelial Cells (“HUVEC”s) grown on gelatin microcarriers and embedded in a fibrin gel. Representative pictures 7 days after HUVEC-seeded microcarriers were cultured in normal conditions (FIG. 1A) or PEMF (FIG. 1B) demonstrate increased tubulization of PEMF. FIG. 1C is a graph showing the number of microcarriers exhibiting tubulization of greater than 1 diameter (>1 MC), 2 diameters (>2 MC), or 3 diameters (>3 MC). Fifty microcarriers were chosen at random. FIG. 1D is a graph showing the number of tubules present on each microcarrier. As illustrated in FIG. 1E, the extent of proliferation of HUVECs over a 48-hour period was examined by light microscopy and quantified by thymidine incorporation (FIG. 1F), revealing that PEMF significantly augmented the proliferation of HUVECs (p<0.01), but had no effect on osteoblasts or fibroblasts (FIG. 1G). FIG. 1H is a graph showing that media cultured in PEMF was able to enhance the proliferation of HUVECs, but denaturing the media ablated this effect. HUVEC proliferation in PEMF was not inhibited by the addition of indomethacin, a prostaglandin (PGE2 ) synthesis inhibitor.

FIGS. 2A-H show PEMF stimulation of the release of Fibroblast Growth Factor 2 (“FGF-2”), but not Vascular Endothelial Growth Factor (“VEGF”), by HUVECs. FIG. 2A is a Northern blot demonstrating no change in VEGF mRNA when HUVECs were grown in PEMF or control conditions. FIG. 2B is a graph showing that VEGF ELISA revealed no differences in VEGF protein production by HUVECs exposed to PEMF. In FIG. 2C, the presence of recombinant VEGF R2/Fc chimera or anti-VEGF antibody is shown to be unable to block the response of HUVEC proliferation to PEMF. In contrast, FGF-2 production was found to be two-fold greater in PEMF conditions. In FIG. 2D, FGF-2 ELISA verified that PEMF stimulated a 3-fold increase in HUVEC production of FGF-2 protein. FIG. 2E is a Northern blot demonstrating an increased degree of FGF-2 transcription in response to PEMF, normalized using GAPDH. FIG. 2F is a graph showing that the addition of FGF-2 neutralizing antibody significantly reduced the degree of stimulation in response to PEMF. Control cultures exhibited no changes in proliferation in response to the antibody. FIG. 2G is a graph showing that media collected from PEMF cultures was able to induce significant (>100% above baseline) proliferation in HUVECs and fibroblasts, but not osteoblasts. The graph in FIG. 2H shows that migration of fibroblasts and HUVECs in response to media collected from PEMF-conditioned HUVEC cultures was 3-fold greater in both cell types compared to cells stimulated with unconditioned media.

FIGS. 3A-E show that PEMF promotes angiogenesis in an in vivo Matrigel plug assay. FIGS. 3A-B show vascular ingrowth within Matrigel 2 weeks after implantation was confirmed by staining for CD31 and Tie2. FIG. 3C shows representative pictures of Matrigel (dotted box) from mice in control and PEMF cages, demonstrating that exposure to PEMF led to an increase in the degree of vascular ingrowth relative to controls. Scale bars represent 25 μm. FIG. 3D shows high power views of a representative section of Matrigel from control mice (top panel) and PEMF-exposed mice (bottom panel). FIG. 3E is a graph showing quantification of cells within the Matrigel, which demonstrates a significant PEMF stimulation of vascular ingrowth at days 3, 10, and 14. Scale bars represent 25 μm.

### DETAILED DESCRIPTION OF THE INVENTION

One aspect of the present invention relates to a method of inducing angiogenesis in a cell or tissue. This method involves applying an electromagnetic field to the cell or tissue under conditions effective to induce angiogenesis.

The process of angiogenesis involves the sprouting of capillaries from existing small vessels. Endothelial cells form new capillaries that grow out from the side of a capillary or small venule by extending long processes or pseudopodia. The cells at first form a solid sprout, which then hollows out to form a tube. This process continues until the sprout encounters another capillary, with which it connects, allowing blood to circulate. Thus, in carrying out the methods of the present invention, angiogenesis is induced in endothelial cells, or tissue comprised of endothelial cells, by applying an electromagnetic field to endothelial cells or tissue comprising endothelial cells under conditions effective to induce angiogenesis.

Application of an electromagnetic field to a cell or tissue is preferably carried out with a device capable of emitting an electromagnetic field. Clinically-approved device technology is widely available (Kanno et al., “Establishment of a Simple and Practical Procedure Applicable to Therapeutic Angiogenesis,” Circulation 99:2682-2687 (1999), which is hereby incorporated by reference in its entirety). Examples of such devices include bone healing devices used for spinal fusion and fracture non-union healing by EBI (Parippany, N.J.) and other orthopedic manufacturers. In a preferred embodiment, the device used in carrying out the methods of the present invention, emits a pulsed electromagnetic field which operates at a frequency of about 5 Hz to 25 Hz, preferably at a frequency of about 15 Hz. In addition, the device produces asymmetric pulses lasting for about 1 msec to 10 msec, preferably for about 4.5 msec.

The device can be incorporated into a dressing such as an Unna Boot, or incorporated into a piece of clothing such as a sock, glove, or jacket. Alternatively, it can be implanted under the skin (which would require either a battery or external power such as occurs with pacemakers) or into an open wound to fill it. For tissue engineering, it would consist of coils which would surround the tissue in which angiogenesis is needed.

Another aspect of the present invention relates to a method of treating an ischemic condition in a patient. This method involves applying an electromagnetic field to ischemic tissue in a patient under conditions effective to treat the ischemic condition by inducing angiogenesis.

Ischemic conditions treatable by the methods of the present invention include, without limitation, coronary artery disease, peripheral vascular disease, cerebrovascular disease, and other conditions in which localized tissue suffers from anemia due to obstruction of blood flow. Thus, the methods of the present invention are also effective in the treatment of wounds, such as a chronic wound or a diabetic wound, by facilitating the process of wound healing.

Application of an electromagnetic field to ischemic tissue is carried out with a device as described supra. The electromagnetic field applied to ischemic tissue may be applied to surgically exposed tissue, or skin overlying tissue. Alternatively, ischemic tissue may be positioned within an electromagnetic field device, where an electromagnetic field is applied to the ischemic tissue.

A patient with an ischemic condition includes any mammal. Preferably the patient is human.

A further aspect of the present invention relates to a method of inducing activity of angiogenic growth factors. This method involves applying an electromagnetic field to a cell or tissue under conditions effective to induce activity of an angiogenic growth factor.

Angiogenic growth factors are well known in the art, and include, without limitation, vascular endothelial growth factors, fibroblastic growth factors, and transforming growth factors. A preferred angiogenic growth factor induced by the method of the present invention is the angiogenic growth factor Fibroblast Growth Factor 2.

Application of electromagnetic fields may also have utility in the field of tissue engineering. A major obstacle preventing the development of techniques to engineer replacements for failing organs is the inability to adequately vascularize tissues created in vitro. Intact organs contain a highly complex three-dimensional network of arterioles, capillaries, and venules, which allow for the efficient exchange of oxygen, nutrients, and metabolic intermediaries. PEMF may be used to promote the formation of a vascularized scaffold to create neo-organs in vitro.

Tissue engineering aims to develop biological substitutes to restore, maintain, or improve tissue function. PEMF is an industrially scaleable technology that can be employed to in vitro tissue engineered constructs to promote vascular ingrowth. Delivery of PEMF can therefore easily be applied to in vivo, ex vivo, or in vitro tissue engineered constructs in order to promote angiogenesis.

Thus, another aspect of the present invention relates to a method of tissue engineering. This method involves providing a tissue scaffold and subjecting the tissue scaffold to an electromagnetic field under conditions effective to form a vascularized tissue scaffold.

Various approaches exist for the construction of tissue scaffolds in the engineering of vascular vessels, which are suitable in carrying out the method of tissue engineering of the present invention. One approach involves adding cells to preformed structures made of biodegradable polymers (Shin'oka et al., “Creation of Viable Pulmonary Artery Autografts through Tissue Engineering,” Journal of Thoracic & Cardiovascular Surgery 115:536-545 (1998); Niklason et al., “Functional Arteries Grown In Vitro,” Science 284:489-493 (1999); Shin'oka et al., “Transplantation of a Tissue-Engineered Pulmonary Artery,” New England Journal of Medicine 344:532-533 (2001); Niklason et al., “Morphologic and Mechanical Characteristics of Engineered Bovine Arteries,” J. Vasc. Surg. 33:628-638 (2001); Hoerstrup et al., “Tissue Engineering of Small Caliber Vascular Grafts,” European Journal of Cardio-Thoracic Surgery 20:164-169 (2001), which are hereby incorporated by reference in their entirety). Another approach is cell entrapment within a biopolymer, which involves the use of gels, typically type 1 collagen, molded into a tube after cells are added to the solution phase prior to gelation (Weinberg and Bell, “A Blood Vessel Model Constructed from Collagen and Cultured Vascular Cells,” Science 231:397-400 (1986); L'Heureux et al., “In Vitro Construction of a Human Blood Vessel from Cultured Vascular Cells: A Morphologic Study,” Journal of Vascular Surgery 17:499-509 (1993), which are hereby incorporated by reference in their entirety). When the cells compact these gels and an appropriate mechanical constraint is applied, it yields a circumferential alignment of fibrils and cells which resemble that of the vascular media (L'Heureux et al., “In Vitro Construction of a Human Blood Vessel from Cultured Vascular Cells: A Morphologic Study,” Journal of Vascular Surgery 17:499-509 (1993); Barocas et al., “Engineered Alignment in Media Equivalents: Magnetic Prealignrment and Mandrel Compaction,” J. Biomech. Eng. 120:660-666 (1998); Seliktar et al., “Dynamic Mechanical Conditioning of Collagen-Gel Blood Vessel Constructs Induces Remodeling In Vitro,” Annals of Biomedical Engineering 28:351-362 (2000); Girton et al., “Confined Compression of a Tissue-Equivalent: Collagen Fibril and Cell Alignment in Response to Anisotropic Strain,” Journal ofBiomechanical Engineering 124:568-575 (2002), which are hereby incorporated by reference in their entirety). Other tissue scaffolds are continually being developed.

### EXAMPLES

The following examples are provided to illustrate embodiments of the present invention, but they are by no means intended to limit its scope.

### Example 1

### Cell Culture

HUVECs (Clonetics, San Diego, Calif.) were cultured in endothelial basal medium (EBM-2) supplemented with EGM-2MV and studied at passages 4-7. Fibroblasts were harvested from newborn foreskin specimens (Freshney, in Culture of Animal Cells: A Manual ofBasic Technique, pgs. 149-175. Wiley-Liss, Inc., New York, 2000, which is hereby incorporated by reference in its entirety). Osteoblasts were harvested from fetal rat calvaria (Steinbrech et al., “VEGF Expression in an Osteoblast-Like Cell Line is Regulated by a Hypoxia Response Mechanism,” Am. J Physiol. Cell. Physiol. 278:C853-60 (2000), which is hereby incorporated by reference in its entirety). Both fibroblasts and osteoblasts were cultured in DMEM supplemented with 10% FBS and 100 μg/ml penicillin G, 50 μg/ml streptomycin and 0.25 μg/ml amphotericin B.

### Example 2

### Exposure to PEMF

Pulsed electromagnetic fields were generated by a bone healing device (EBI, Parsippany, N.J.) delivering uniform time-varying fields. Fields consisted of asymmetric 4.5 msec pulses repeated at 15 Hz, with a magnetic flux density rising from 0 to 12 gauss in 200 μsec and returning to 0 G in 25 μsec. PEMF generators were placed inside identical incubators, but only turned on in the test incubator. Extraneous 50 Hz magnetic fields within each incubator were less than 2 mG. Custom designed cages surrounded with the same configuration were employed for the in vivo experiments.

### Example 3

### In Vitro Angiogenesis Assay

A microcarrier (“MC”) in vitro angiogenesis assay was performed as previously described (Nehls et al., “A Novel, Microcarrier-Based In Vitro Assay for Rapid and Reliable Quantification of Three-Dimensional Cell Migration and Angiogenesis,” Microvasc. Res. 50:311-322 (1995), which is hereby incorporated by reference in its entirety). HUVECs were added to a suspension of MCs (Cytodex 3®), and cultured until confluent. Fibrin gels were prepared by dissolving fibrinogen (Sigma, St. Louis, Mo.) in PBS (2.5 mg/ml) along with 200 U/ml of aprotinin to prevent excessive fibrinolysis. Confluent HUVEC-seeded MCs were added to each well and polymerization was achieved at 1 hour by adding thrombin (0.625 U/ml). Gels were cultured in the presence or absence of PEMF for 7-10 days. The degree of angiogenesis was quantified by two blinded observers assessing 50 MCs at random and counting: (1) the number of MCs with tubules greater than one, two, or three MC diameters and (2) the exact number of tubules on each MC.

### Example 4

### Proliferation Assay

HUVECs (1×10⁵) seeded onto 6-well plates were cultured for 24 hours with EBM+1% FBS (starved media). The media was then changed to fully supplemented media, at which time cultures were separated into their respective incubators for an additional 24 hours. A 24-hour proliferation assay was performed by the addition of 5 μCi of radioactive thymidine [³H] three hours prior to the completion of the assay. Cells were washed with PBS×3 and 10% trichloroacetic acid×3, followed by the addition of 2 mL of 1N NaOH for 30 minutes and neutralizations with 2 mL 1N HCl. Independent cell cultures were used for each experiment (n=6), run in triplicate, and evaluated using a scintillation counter. A subset of HUVEC cultures (n=3) were trypsinized and the number of cells/well was counted manually with a hematocytometer after 24 hour exposure to PEMF.

For fibroblast and osteoblast proliferation studies, 1×10⁵ cells were seeded on 6-well plates in starved DMEM (with 1% FBS) and replaced with media collected from HUVEC cultures after 24 hours PMF exposure. After 24 hours, proliferation was measured by thymidine incorporation as described supra.

### Example 5

### Migration Assay

Migration was studied using a modified transwell assay. HUVECs and fibroblasts in starvation media (EBM or DEMM+1% FBS) were seeded onto Chemo Tx® filters (5.7 mm, 8 μm pore size) (Neuro Probe, Gaithersburg, Md.). Media (either stock EBM, DMEM, or collected from HUVEC cultures after 24 hours PEMF exposure) was added to the lower chamber. After 24 hours incubation, non-migrating cells were completely wiped from the top surface of the membrane and migrating cells adherent to the underside of the filter were quantified using the nuclear dye DAPI (Vector Labs, Burlingame, Calif.) and fluorescent microscopy.

### Example 6

### Media Denaturing Experiments

Media from HUVEC cultures in the absence or presence of PEMF was collected as donor media (conditioned media). In addition, PEMF and normal-conditioned media were heated at 100° C. for 20 minutes and immediately cooled on ice for 20 minutes. The denatured media was resupplemented with EGM-2MV to replace essential growth factors that were also denatured, and then similarly used as growth media for a HUVEC proliferation assay. Results were normalized to the thymidine incorporation observed in cultures receiving media harvested from HUVECs not exposed to PEMF (n=6).

### Example 7

### Prostaglandin Synthesis Inhibition

A 48 hour thymidine incorporation assay was performed in the presence of indomethacin (Calbiochem, EMD Biosciences, San Diego, Calif.), a phospholipase A₂ inhibitor (Fitzpatrick et al., “Cytochrome P-450 Metabolism of Arachidonic Acid: Formation and Biological Actions of ‘Epoxygenase-Derived’ Eicosanoids,” Pharmacol. Rev. 40:229-241 (1988), which is hereby incorporated by reference in its entirety), at a concentration of 4 μg/ml, previously shown to completely block prostaglandin synthesis in vitro (n=4) (Brighton et al., “Signal Transduction in Electrically Stimulated Bone Cells,” J. Bone Joint Surg. Am. 83-A:1514-1523 (2001), which is hereby incorporated by reference in its entirety).

### Example 8

### VEGF ELISA and Northern Blot Analysis

A mouse VEGF sandwich enzyme immunoassay (R&D Systems, Minneapolis, Minn.) was used to measure the quantity of VEGF (165-amino acid isoform) in media from PEMF and control cultures (n=4; run in triplicate). For Northern blot analysis, total cellular RNA was extracted by cell lysis (TRIzol). RNA (20 μg) was separated on a 1% agarose containing 2.0 M formaldehyde and transferred to a Brightstar-Plus nylon blotting membrane (Ambion, Austin, Tex.) via Turbo Blot downward transfer system. RNA was crosslinked via the UV Stratalinker 1800 (Stratagene, La Jolla, Calif.) and hybridized with VEGF and 18S cDNA probes labeled with P³²-dCTP (Amersham Biosciences, Piscataway, N.J.) (Steinbrech et al., “VEGF Expression in an Osteoblast-Like Cell Line is Regulated by a Hypoxia Response Mechanism,” Am. J. Physiol. Cell Physiol. 278. C853-860 (2000), which is hereby incorporated by reference in its entirety). Band densitometry was performed using Kodak ID.

### Example 9

### VEGF Blocking Assays

PEMF- and control-conditioned media were used for a 48-hour HUVEC proliferation assay and supplemented with either 0.1 mg/mL of anti-human VEGF antibody or 50 ng/mL of recombinant human VEGF R2(KDR)/Fc chimera (R&D Systems, Minneapolis, Minn.), concentrations previously shown to eliminate soluble VEGF activity (n=4; run in triplicate) (Millauer et al., “High affinity VEGF Binding and Developmental Expression Suggest Flk-1 as a Major Regulator of Vasculogenesis and Angiogenesis,” Cell 72:835-846 (1993), which is hereby incorporated by reference in its entirety).

### Example 10

### Angiogenic Protein Screening

PEMF and control conditioned media was harvested after 48 hours of incubation and analyzed via a sandwich ELISA assay (SearchLight Angiogenesis Array; Pierce Technologies, Boston, Mass.). Media samples (50 μL) were incubated for 1 hour in ELISA coated with antibodies to angiogenic proteins; tissue inhibitor of matrix metalloproteinase-1 (TIMP-1), angiopoietin-2 (ang-2), platelet-derived growth factor (PDGF), thrombopoietin (TPO), keratinocyte growth factor (KGF), hepatocyte growth factor (HGF), and epidermal growth factor (EGF). Total concentrations (pg/ml) were determined through chemiluminescent signaling. All experiments (n=3) were done in triplicate.

### Example 11

### FGF-2 ELISA and Northern Blot

A mouse FGF-2 sandwich enzyme immunoassay (R&D Systems, Minneapolis, Minn.) was used to measure the quantity of FGF-2 in media from PEMF and control cultures (n=4; experiments run in triplicate). For Northern blot analysis (n=3), total cellular RNA was extracted by cell lysis (TRIzol). RNA (50 μg) was separated on a 1% agarose containing 2.0 M formaldehyde and transferred to a Brightstar-Plus nylon blotting membrane (Ambion, Austin, Tex.) via Turbo Blot downward transfer system. RNA was crosslinked via the UV Stratalinker 1800 (Stratagene, La Jolla, Calif.) and hybridized with FGF-2 and 18S cDNA probes labeled with P³²-dCTP (Amersham Biosciences, Piscataway, N.J.) (Steinbrech et al., “VEGF Expression in an Osteoblast-Like Cell Line is Regulated by a Hypoxia Response Mechanism,” Am. J. Cell Physiol. 278:C853-860 (2000), which is hereby incorporated by reference in its entirety). Band densitometry was performed using Kodak ID.

### Example 12

### FGF-2 Blocking Assays

FGF-2 neutralizing antibody (donated by Dr. David A. Moscatelli, New York, N.Y.) was added to cultures also receiving either PEMF- or control-conditioned media (Sato et al., “Autocrine Activities of Basic Fibroblast Growth Factor: Regulation of Endothelial Cell Movement, Plasminogen Activator Synthesis, and DNA Synthesis,” J. Cell Biol. 107:1199-1205 (1988), which is hereby ncorporated by reference in its entirety). The FGF-2 antibody was added each time he media was changed, and a thymidine incorporation assay was performed after 48 hours (n=6; each experiment was run in triplicate).

### Example 13

### In Vivo Matrigel Plug Assay

All experiments were performed in full accordance with the NYU Medical Center Institutional Animal Care and Use Committee. Tie2/lacZ mice (Jackson Laboratory; 10-16 weeks) received a subcutaneous injection with Matrigel supplemented with basic-FGF-2 (25 ng). Mice were housed in either control cages (n=8) or in cages that delivered PEMF (n=10) consecutively for 8 hours per day. Matrigel samples were snap frozen or fixed in 4% paraformaldehyde and tissue sections were stained histochemically with x-gal solution overnight at 4° C., or immunohistochemically with rat anti-mouse CD31 (Becton Dickinson, Franklin Lakes, N.J.), and Alexa-Flour goat anti-rat secondary antibody (Molecular Probes, Eugene, Oreg.). The number of nuclei contained per high power field (hpf) of Matrigel was counted in 20 random fields by two blinded observers. For ELISA analysis, Matrigel plugs were harvested and submerged in T-PER Extraction Reagent (Pierce/Perbio, Inc., Rockford, Ill.) with 100 μl/ml protease inhibitor added, mechanically homogenized, and centrifuged. Supernatant was removed and assayed using the ELISA protocol described previously for FGF-2. Similar sandwich enzyme immunoassay kits (R&D Systems, Minneapolis, Minn.) were used to assay TPO, ang-2, and EGF (n=4; experiments run in triplicate).

### Example 14

### Statistical Analysis

Statistical analysis was calculated based on a 2-tailed t-test and all data are presented as mean±S.E.M. A p<0.05 was considered statistically significant.

### Example 15

### PEMF Induces Endothelial Tubule Formation

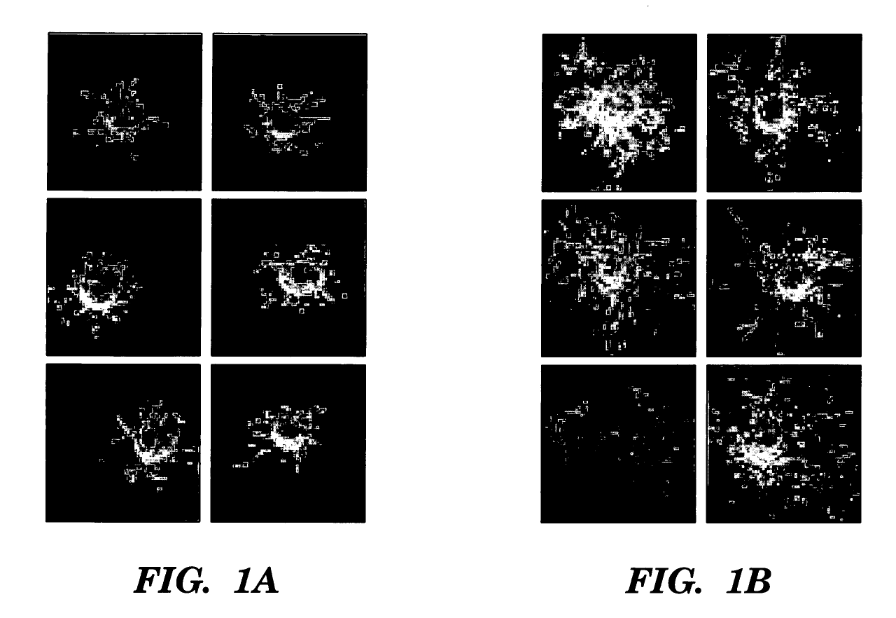

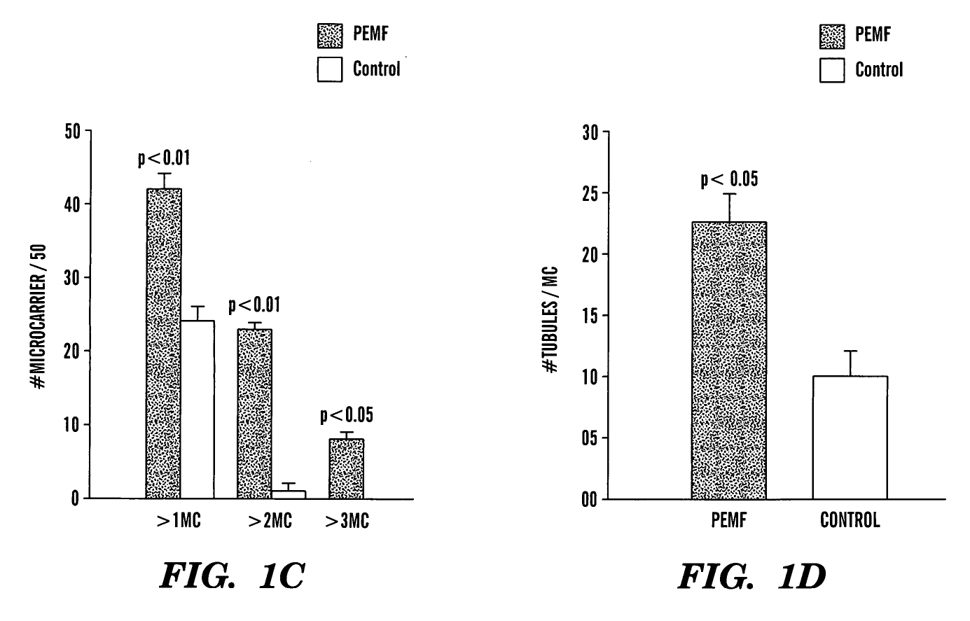

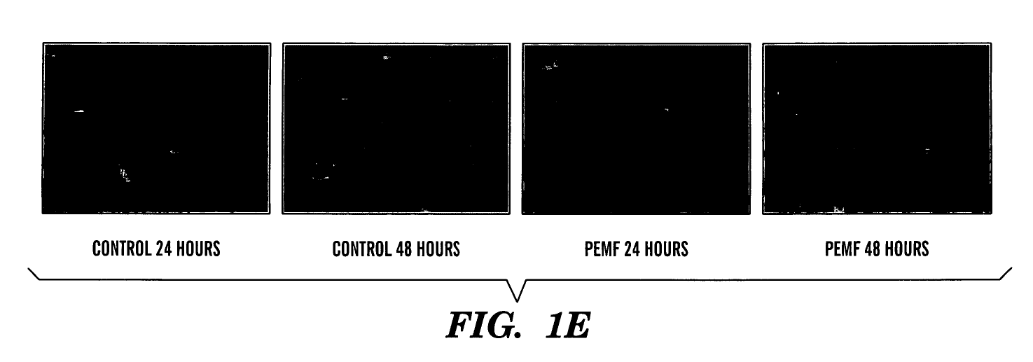

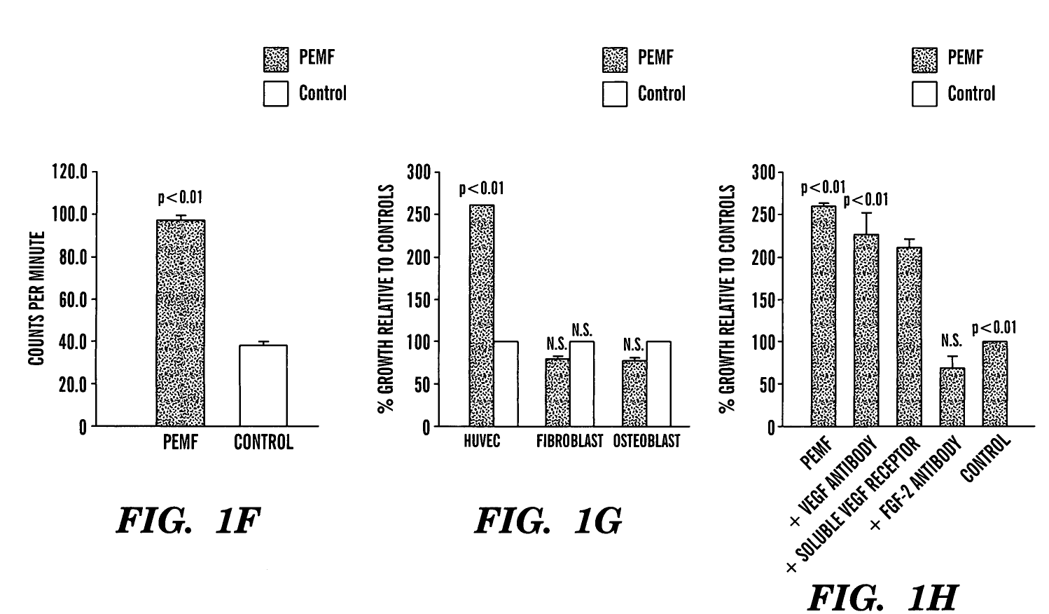

The gelatin microcarrier assay is a well established in vitro model of angiogenesis and quantifies the ability of endothelial cells to sprout from a single focus (Nehls et al., “A Novel, Microcarrier-Based In Vitro Assay for Rapid and Reliable Quantification of Three-Dimensional Cell Migration and Angiogenesis,” Microvasc. Res. 50:311-22 (1995), which is hereby incorporated by reference in its entirety). Human umbilical vein endothelial cells grown on microcarriers in the absence (FIG. 1A) or presence (FIG. 1B) of PEMF demonstrated a substantial increase in tubulization. Tubulization was quantified in two ways: (1) the total fraction of MCs with a tubule length greater than one, two, or three diameters and (2) the total number of tubules on each microcarrier. In PEMF, a several fold increase in tubulization of one or two diameters was seen (41/50 vs 24/50, 21/50 vs 3/50; p<0.01) (FIG. 1C), and only cells exposed to PEMF developed tubules greater than three diameters (6/50 vs 0/50; p<0.05). Exposure to PEMF also led to a significant increase in the total number of tubules per microcarrier (2.25±0.45 vs 1.00±0.25; p<0.05) (FIG. 1D).

### Example 16

### PEMF Stimulates Endothelial Proliferation

Thymidine incorporation established that HUVECs exposed to PEMF demonstrated enhanced proliferation compared to controls (9.2×10⁴ vs 3.5×10⁴ cpm; p<0.01) (FIGS. 1E-G). This increase in proliferation correlated with an increase in absolute cell number (220±14×10³ cells/well in PEMF vs 117±9×10³ cells/well in controls) over the 24 hour course of the experiments. Fibroblast and osteoblast cell lines, under identical conditions of PEMF exposure, did not exhibit any change in thymidine incorporation or cell number (p=0.23 and p=0.29, respectively).

### Example 17

### PEMF Releases a Soluble Pro-Angiogenic Protein FGF-2

Media harvested from HUVECs cultured in PEMF (PEMF-conditioned media) increased proliferation of HUVECs not directly exposed to PEMF, suggesting a soluble factor was responsible. The average HUVEC response to PEMF-conditioned media was two-fold greater than HUVECs given media from control cells not exposed to PEMF (222.0±2.3%; p<0.01). The addition of a cyclooxygenase inhibitor (indomethacin) was unable to block PEMF-induced stimulation (206.0±2.7%; p<0.01), suggesting that arachadonic acid metabolites were not involved. In contrast, heat denaturing eliminated the stimulatory effects of PEMF-conditioned media on HUVECs (77.8±10.2% vs 222.0±2.3%; p<0.01), demonstrating that a soluble protein was responsible for the proliferative activity (FIG. 1H).

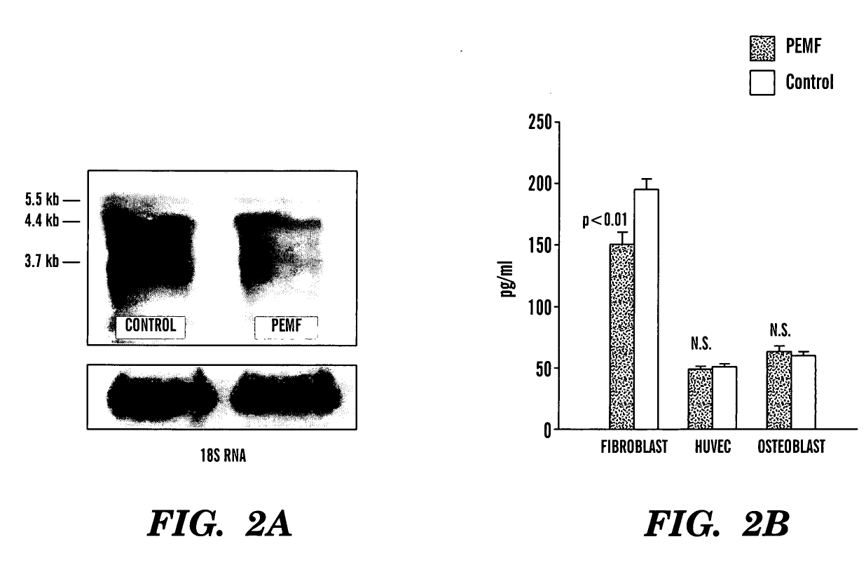

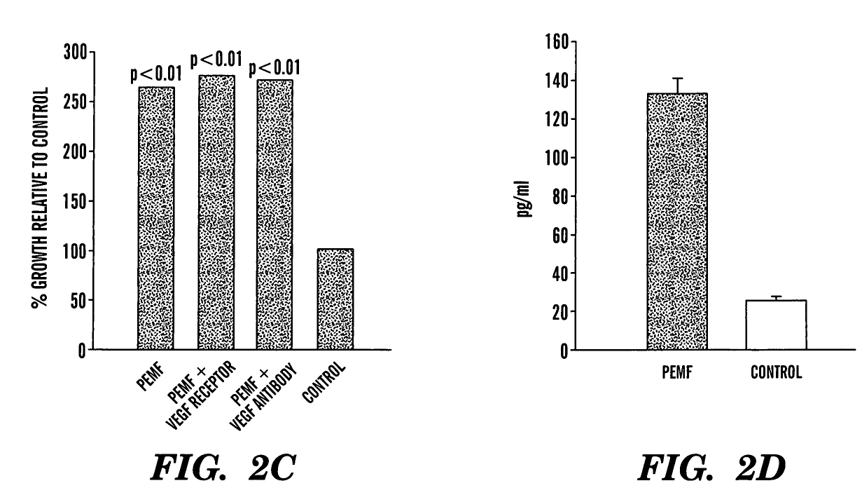

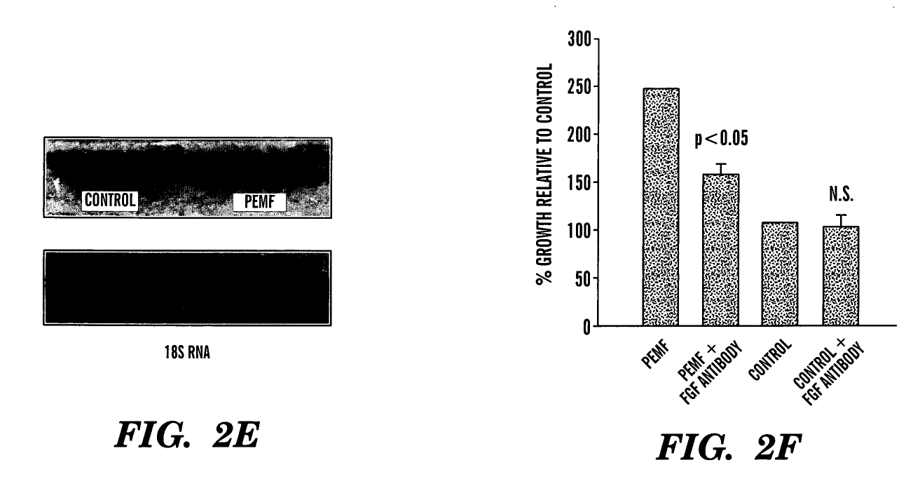

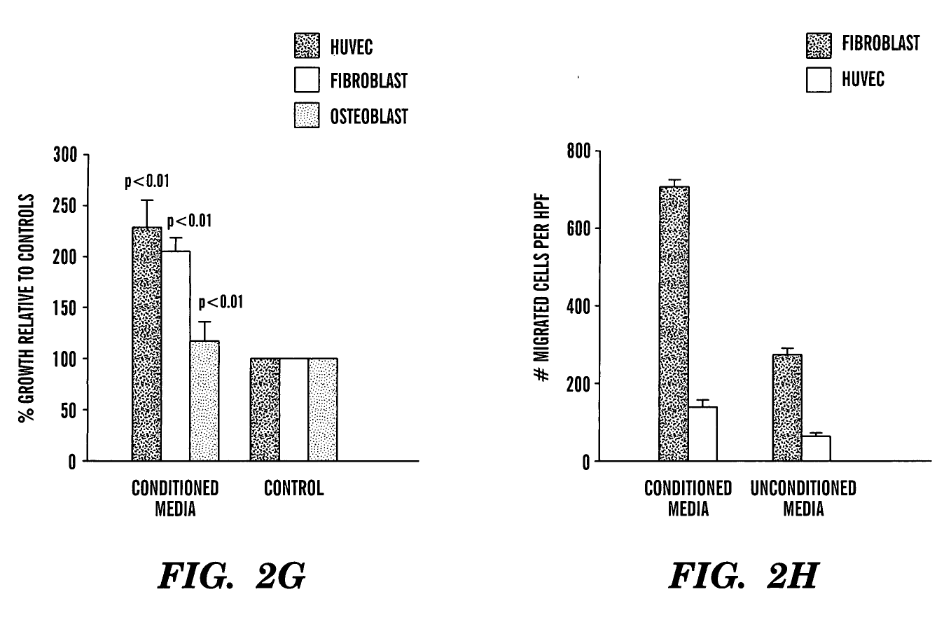

The most likely candidate responsible for pro-angiogenic effect is VEGF, a potent vascular mitogen (Losordo et al., “Gene Therapy for Myocardial Angiogenesis: Initial Clinical Results with Direct Myocardial Injection of phVEGF165 as sole therapy for myocardial ischemia,” Circulation 98:2800-2804 (1998), which is hereby incorporated by reference in its entirety). However, no differences were observed in VEGF-A mRNA or protein levels within PEMF cultures when compared to controls (mean intensity 167.57 vs 172.23, 51.25±4.98 pg/ml vs 50.79±3.78 pg/ml, respectively; p=0.81) (FIGS. 2A and B). To further confirm that VEGF signaling was not involved, proliferation assays were also performed in the presence of anti-VEGF antibody or recombinant VEGF-receptor 2 (KDR)/Fc chimera, both potent blockers of soluble VEGF activity. HUVEC proliferation in response to PEMF was unchanged in the presence of these blocking agents (284.8±17.3% and 266.1±10.0% vs 222.0±2.3% with conditioned-media alone, respectively) (FIG. 2C).

Since VEGF is only one of many potential angiogenic factors, angiogenic protein screening of PEMF conditioned media was performed. Protein concentrations for tissue inhibitor of matrix metalloproteinase-1 (TIMP-2), platelet-derived growth factor (PDGF), and hepatocyte growth factor (HGF) were not significantly altered in PEMF conditions versus controls. In contrast, FGF-2 production was found by ELISA to be increased five-fold by exposure to PEMF (FIG. 2D). Northern blot analysis also revealed an increase in FGF-2 mRNA in cultures incubating in PEMF (mean intensity 975635 vs 651316; p<0.05) (FIG. 2E). The addition of FGF-2 neutralizing antibody inhibited the stimulatory effects of PEMF on HUVEC proliferation, but did not return it to baseline (147.28%±9.73% vs 94.85% ±3.70%, p<0.05) (FIG. 2F). Additional proteins with smaller significant elevations were angiopoietin-2 (ang-2), thrombopoietin (TPO), and epidermal growth factor (EGF): (2320.3±1128.4 vs 3323.8±1168.7 pg/ml; p<0.05), (46.7±4.3 vs 133.1±51.4 pg/ml; p<0.05), and (4.8±1.3 vs 7.1±0.4 pg/ml; p<0.05), respectively.

### Example 18

### Conditioned Media Stimulates Proliferation in Fibroblasts, but not Osteoblasts

Under direct stimulation with PEMF, HUVECs proliferated exponentially and released significant amounts of FGF-2. However, fibroblast and osteoblast proliferation did not increase appreciably after PEMF exposure. To determine whether paracrine FGF-2 signaling occurred from HUVECs to parenchymal tissues, fibroblast and osteoblast proliferation was studied under the influence of media collected from HUVEC cultures after 24 hours of PEMF exposure. Using the same thymidine assay described previously, 24 hours of exposure to conditioned media resulted in a significant (>100%) increase in fibroblast growth when compared to controls. However, osteoblast proliferation did not change significantly under the same conditions (FIG. 2G).

### Example 19

### Conditioned Media Stimulates Fibroblast and HUVEC Migration

To further confirm the importance of FGF-2 signaling from HUVECs and examine functional cell changes induced by protein release, fibroblast and HUVEC migration was studied using PEMF-conditioned HUVEC media as a chemotactic agent. The migratory populations of both fibroblasts and HUVECs more than doubled under the influence of PEMF-conditioned media (FIG. 2H).

### Example 20

### PEMF Stimulates In Vivo Angiogenesis

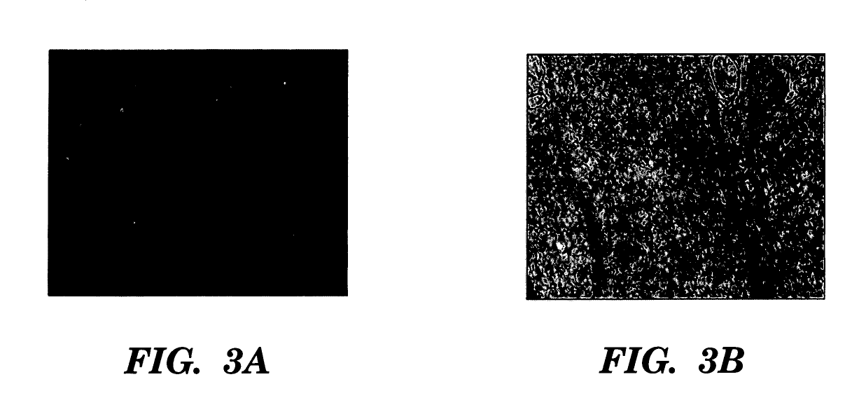

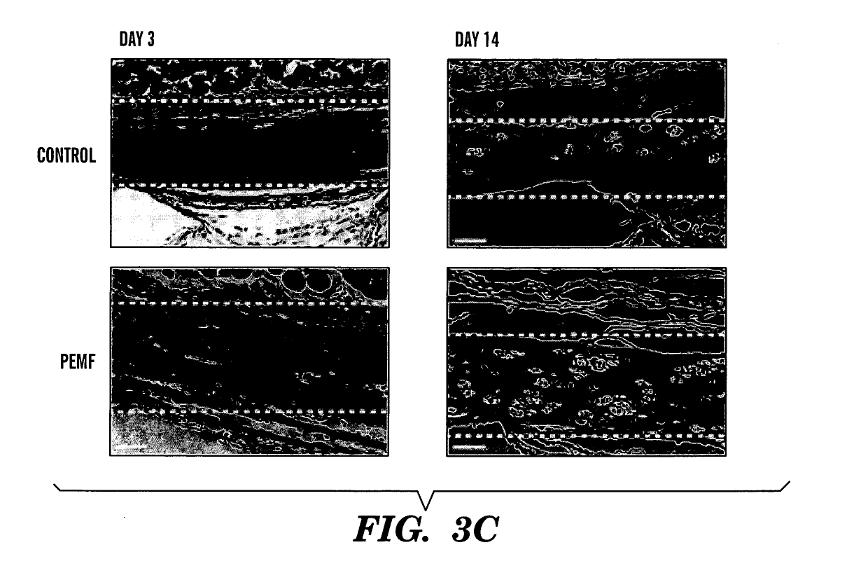

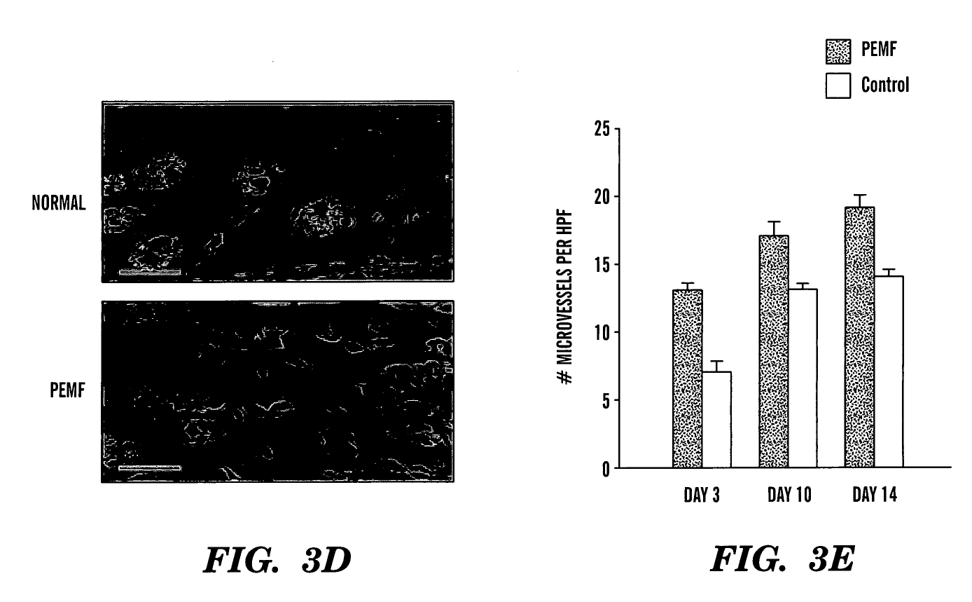

Having demonstrated that PEMF has a potent effect on endothelial cells in vitro, it was determined whether PEMF was able to stimulate angiogenesis in vivo. Matrigel is a soluble basement membrane preparation, and when implanted subcutaneously, supports vascular ingrowth. Matrigel was injected subcutaneously into tie2/lacZ transgenic mice that were housed in cages emitting PEMF for 8 hours a day or control cages. After 3, 10, and 14 days, there was significantly greater vascular ingrowth into the matrix in PEMF-treated animals, confirmed by staining specific for endothelial markers CD31 and Tie-2. PEMF increased the vascular ingrowth more than 2-fold by day 3 (13.3±0.41 vs 5.8±0.28 cells/hpf; p<0.01). This increase in vascular ingrowth persisted through days 10 and 14 (16.6±0.49 vs 12.6±0.43 cells/hpf; p<0.01, and 19.4±0.55 vs 14.8±0.40 cells/hpf; p<0.01, respectively) (FIG. 3). ELISA confirmed a 2-fold increase in FGF-2 in PEMF-treated Matrigel, but demonstrated no differences in the growth factors TPO, ang-2, and EGF.

PEMF is shown herein to stimulate processes critical for angiogenesis. The delivery of PEMF at low doses, identical to that currently in clinical use, significantly increased endothelial cell proliferation and tubulization, processes important for vessel formation. The ability of PEMF to increase cellular proliferation was unique to endothelial cells, while the addition of media from conditioned HUVECs to both fibroblast and HUVEC cultures increased proliferation and migration. This suggests that endothelial cells are the primary target for PEMF stimulation, releasing protein in a paracrine fashion to induce changes in neighboring cells, and upregulating angiogenesis. However, both direct stimulation and conditioned media studies revealed no significant change in osteoblast proliferation. Thus, the ability of PEMF to enhance the healing of complicated fractures is likely the result of increased vascularity rather than a direct effect on osteogenesis as previously believed.

While VEGF is the most ubiquitous mediator of angiogenesis, it was not responsible for the angiogenic effect of PEMF in the experiments described herein. Angiogenic protein screening demonstrated a five-fold increase in FGF-2, a well described angiogenic mediator. While the addition of an FGF-2 neutralizing antibody reduced PEMF-stimulation of endothelial cells, proliferation did not return completely to baseline. It is therefore possible that PEMF does not simply act through the upregulation of a single agent (i.e., FGF-2), but involves the coordinated release of other angiogenic proteins or cytokines. However, only significant increases in FGF-2 were detected in vivo. Thus, it seems likely that FGF-2 signaling is the predominant mechanism, and these cytokine changes are secondary. The in vitro potency of PEMF to increase endothelial cell proliferation was comparable to that of high doses of VEGF or FGF, suggesting that this phenomenon is of true biologic relevance in vivo (Bernatchez et al., “Relative Effects of VEGF-A and VEGF-C on Endothelial Cell Proliferation, Migration, and PAF Synthesis: Role of Neuropilin-1,” J. Cell Biochem., 85:629-639 (2002), which is hereby incorporated by reference in its entirety).

To support this, the effect of PEMF on in vivo angiogenesis was examined. Using the well-established Matrigel assay, it was demonstrated that PEMF was able to significantly increase angiogenesis in vivo. Recent evidence suggests that blood vessels in the adult may result from either expansion of existing endothelial cells, or the recruitment of bone marrow-derived endothelial progenitor cells (“EPC”s) (Isner et al., “Angiogenesis and Vasculogenesis as Therapeutic Strategies for Postnatal Neovascularization,” J. Clin. Invest. 103:1231-1236 (1999), which is hereby incorporated by reference in its entirety). Although the effects of PEMF on bone marrow derived EPCs were not directly assessed, the in vitro data on fully differentiated endothelial cells indicates that the effects of PEMF are directed towards pre-existing endothelial cells.

If PEMF is able to augment angiogenesis, its clinical utility may extend well beyond its current role in bone healing. One application is in the field of therapeutic angiogenesis, defined as the artificial manipulation of blood vessel growth for the treatment of ischemic conditions. The majority of existing techniques for therapeutic angiogenesis are based on the delivery of single pro-angiogenic cytokines or the supplementation of vascular stem cells (Isner et al., “Angiogenesis and Vasculogenesis as Therapeutic Strategies for Postnatal Neovascularization,” J. Clin. Invest. 103:1231-1236 (1999), which is hereby incorporated by reference in its entirety). Agents such as VEGF or FGF have shown promise in animal models, but clinical trials have been disappointing (Carmeliet, “VEGF Gene Therapy: Stimulating Angiogenesis or Angioma-Genesis?” Nat. Med. 6:1102-1103 (2000), which is hereby incorporated by reference in its entirety). Furthermore, difficulties related to immunogenicity, dosing, and means of delivery have limited the widespread clinical impact of these modalities. PEMF may offer distinct advantages as a non-invasive and targeted modality which is able to release several growth factors to achieve therapeutic angiogenesis. Moreover, since PEMF utilizes commonly available, clinically-approved technology, it may have rapid applicability in the treatment of ischemic conditions (Kanno et al., “Establishment of a Simple and Practical Procedure Applicable to Therapeutic Angiogenesis,” Circulation 99:2682-2687 (1999), which is hereby incorporated by reference in its entirety). Data from this study provides a rational basis for use in these conditions.

The finding that PEMF was able to stimulate endothelial cell kinetics raises important questions regarding the relationship between PEMF and carcinogenesis. A number of epidemiological studies have suggested a link between electromagnetic fields and malignancies, including breast cancer, brain cancer, and leukemia (Stix, “Closing the Book. Are Power-Line Fields a Dead Issue?” Sci. Am. 278:33-34 (1998), which is hereby incorporated by reference in its entirety), but the precise mechanism, if any, remains unknown. Although there are multiple papers confirming that electromagnetic fields are not directly mutagenic or carcinogenic, none have examined the possibility that electromagnetic fields may promote tumor progression once malignant transformation has occurred. Since angiogenesis is believed to be essential for tumor growth, spread, and eventual clinical disease, the present study suggests that the link between electromagnetic fields and cancer may be through increased angiogenesis. Epidemiological studies suggest that exposure to PEMF (i.e., high tension power lines) at a wide range of frequencies can be correlated with an increased risk of cancer (Savitz et al., “Leukemia and Occupational Exposure to Electromagnetic Fields: Review of Epidemiologic Surveys,” J. Occup. Med. 29:47-51 (1987), which is hereby incorporated by reference in its entirety). However, the direct comparison to the field strength used in this study is difficult given the wide amplitude window produced by pulsed delivery. Although clinical data suggests that PEMF is safe, the possibility that electromagnetic fields are not themselves carcinogenic, but promote tumor progression via increased angiogenesis warrants further investigation.

In conclusion, although PEMF has been employed for years by clinicians to supplement bone healing, its precise mechanism of action has not been determined. The experiments described herein provide evidence to support the concept that PEMF acts by promoting angiogenesis through the coordinated release of FGF-2, and to a lesser extent, several other vascular growth factors (Ang-2, TPO, and EGF). Thus, PEMF may facilitate healing by augmenting the interaction between osteogenesis and blood vessel growth. This finding not only elucidates a novel mechanism for PEMF action, but also suggests extended applications for PEMF in the treatment of ischemic disease and a potential linkage between electromagnetic fields and tumor biology.

### Example 21

### Accelerated Wound Healing by Pulsed Electromagnetic Fields

While there is evidence of the beneficial effects of PEMF in bone healing, the mechanism of action remains unclear, but may involve increased angiogenesis. Wound healing has not been rigorously examined. This study utilized a diabetic wound model to examine the effects of PEMF on soft tissue healing. Also described are changes in endothelial cell protein secretion indicative of enhanced angiogenesis following PEMF exposure.

Five (5) mm circular wounds were created on the dorsum of db/db and wild type C57BL6 mice, splinted open and covered with an occlusive dressing. Mice were exposed to a clinical bone healing PEMF signal (4.5 ms burst duration/15 Hz) 8 hours/day for 14 days. Gross closure was assessed with digital analysis of area changes over time. Histological examination assessed granulation and epithelial gap, cell proliferation (BrdU), and endothelial cell density (CD31). HUVECs were incubated in the presence or absence of PEMF for 8 hours, and growth factors were measured in culture supernatants by ELISA.

Diabetic mice exposed to PEMF had accelerated wound closure at day 7 (wound area as % of original, PEMF: 60% vs control: 78%, p<0.05) and day 14 (PEMF: 21% vs control: 55%, p<0.05). Because wild-type mice heal twice as fast as diabetics, wounds were analyzed on days 4 and 8. Accelerated closure was evident in PEMF wild-type mice at day 4 (PEMF: 15% vs 42%, p<0.05) and day 8 (8% vs 28%, p<0.05). In wound bed histological sections, granulation and cell proliferation were both increased in PEMF treated diabetic mice (day 7: 52±8 vs 31±5 cells per high power field (200×)). Immunohistochemical analysis revealed significantly higher CD31 density in diabetic wounds exposed to PEMF at day 7 (PEMF: 28±4 vs control 17±4 vessels per high power field) and day 14 (PEMF: 32±6 vs control: 21±5). Increases were also seen in wild-type C57BL6 mice at day 7 (PEMF: 41±7 vs control: 28±6) and day 14 (PEMF: 48±5 vs control: 40±5). HUVECs cultured in PEMF exhibited 5-fold higher levels of FGF2 compared to controls after as little as 30 minutes (20.50 pg/ml±6.75 vs 4.25 pg/ml±0.75), with no change in VEGF.

These findings indicate PEMF accelerates wound closure and increases endothelial cell proliferation. The observed release of FGF2 may account for the increased vascular density and accelerated wound closure, and may also contribute to the beneficial effects of PEMF on bone healing. Other uses of PEMF may include treatment of diabetic ulcers and other non-healing wounds. Other opportunities may exist in aging (since vascular density is known to decrease with age), ischemic pre- conditioning, and tissue engineering.

Although the invention has been described in detail for the purpose of illustration, it is understood that such detail is solely for that purpose, and variations can be made therein by those skilled in the art without departing from the spirit and scope of the invention which is defined by the following claims.

1. A method of inducing angiogenesis in a cell or tissue, said method comprising:
applying an electromagnetic field to the cell or tissue under conditions effective to induce angiogenesis.

2. The method according to claim 1, wherein the cell is an endothelial cell or the tissue comprises endothelial cells.

3. The method according to claim 1, wherein the electromagnetic field comprises pulsed electromagnetic fields.

4. The method according to claim 1, wherein said applying an electromagnetic field is carried out with a device capable of emitting an electromagnetic field.

5. The method according to claim 4, wherein the device capable of emitting an electromagnetic field is a pulsed electromagnetic field device which operates at a frequency of about 5 to 25 Hz.

6. The method according to claim 4, wherein the device capable of emitting an electromagnetic field is a pulsed electromagnetic field device which produces asymmetric pulses lasting for about 1 to 10 msec.

7. A method of treating an ischemic condition in a patient, said method comprising:
applying an electromagnetic field to ischemic tissue in a patient under conditions effective to treat the ischemic condition by inducing angiogenesis.

8. The method according to claim 7, wherein the electromagnetic field comprises pulsed electromagnetic fields.

9. The method according to claim 7, wherein the ischemic condition is coronary artery disease.

10. The method according to claim 7, wherein the ischemic condition is peripheral vascular disease.

11. The method according to claim 7, wherein the ischemic condition is cerebrovascular disease.

12. The method according to claim 7, wherein the ischemic condition is a wound.

13. The method according to claim 12, wherein the wound is a chronic wound.

14. The method according to claim 12, wherein the wound is a diabetic wound.

15. The method according to claim 7, wherein the electromagnetic field is applied to surgically exposed tissue.

16. The method according to claim 7, wherein the electromagnetic field is applied to skin overlying tissue.

17. The method according to claim 7, wherein the ischemic tissue is positioned within an electromagnetic field device.

18. The method according to claim 7, wherein said applying comprises:
providing a device capable of emitting an electromagnetic field, and subjecting the ischemic tissue to an electromagnetic field with said device.

19. The method according to claim 18, wherein the device capable of emitting an electromagnetic field is a pulsed electromagnetic field device which operates at a frequency of about 5 to 25 Hz.

20. The method according to claim 18, wherein the device capable of emitting an electromagnetic field is a pulsed electromagnetic field device which produces asymmetric pulses lasting for about 1 to 10 msec.

21. The method according to claim 7, wherein the patient is human.

22. A method of tissue engineering comprising:
providing a tissue scaffold, and subjecting the tissue scaffold to an electromagnetic field under conditions effective to form a vascularized tissue scaffold.

23. The method according to claim 22, wherein the electromagnetic field comprises pulsed electromagnetic fields.

24. The method according to claim 22, wherein said subjecting is carried out in vivo.

25. A method of inducing activity of angiogenic growth factors, said method comprising:
applying an electromagnetic field to a cell or tissue under conditions effective to induce activity of an angiogenic growth factor.

26. The method according to claim 25, wherein the electromagnetic field comprises pulsed electromagnetic fields.

27. The method according to claim 25, wherein said applying comprises:
applying the electromagnetic field with a device capable of emitting an electromagnetic field.

28. The method according to claim 27, wherein the device capable of emitting an electromagnetic field is a pulsed electromagnetic field device which operates at a frequency of about 5 to 25 Hz.

29. The method according to claim 27, wherein the device capable of emitting an electromagnetic field is a pulsed electromagnetic field device which produces asymmetric pulses lasting for about 1 to 10 msec.

30. The method according to claim 25, wherein the angiogenic growth factor is Fibroblast Growth Factor 2.
